SEQUENCE LISTING (4 sequences)

SEQ ID NO: 1 - protein, 119 aa, Mus musculus [sequence omitted]

SEQ ID NO: 2 - protein, 106 aa, Mus musculus [sequence omitted]

SEQ ID NO: 3 - protein, 25 aa, Homo sapiens
DTHFPICIFCCGCCHRSKCGMCCKT

SEQ ID NO: 4 - protein, 183 aa, Homo sapiens
VPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

(19) United States
(12) Patent Application Publication
(10) Pub. No.: US 2011/0059080 A1
(43) Pub. Date: Mar. 10, 2011

(54) USE OF AN ANTI-IL6 ANTIBODY TO DECREASE HEPCIDIN IN CANCER PATIENTS

(76) Inventors: MARK CORNFELD, RARITAN, NJ (US); MANJULA REDDY, RADNOR, PA (US); XIANG QIN, WAYNE, PA (US); BRITTE RIJNBEEK, LEIDEN (NL)

(21) Appl. No.: 12/876,281

(22) Filed: Sep. 7, 2010

Related U.S. Application Data
(60) Provisional application No. 61/240,363, filed on Sep. 8, 2009.

(51) Int. Cl. A61K 39/395; A61P 35/00; A61P 3/04; A61P 3/00; A61P 7/06; A61P 43/00

(52) U.S. Cl. 424/133.1; 424/158.1; 424/145.1

(57) ABSTRACT

A method of reducing serum hepcidin in a patent having a malignancy or lymphoproliferative disorder by administering an IL-6 neutralizing antibody.

This application claims priority to U.S. Provisional Application Ser. No. 61/240,363, filed on Sep. 8, 2009.

### BACKGROUND OF THE INVENTION

1. Field of the Invention

The invention relates to methods of treating cancer patients wherein the patient exhibits reduced hemoglobin levels and elevated hepcidin levels using an anti-IL6 antibody.

2. Description of the Related Art

There are at least two major biological functions of IL-6: mediation of acute phase proteins, including C-reactive protein (CRP), and acting as a differentiation and activation factor. Acute phase proteins are known to regulate immune responses, mediate inflammation, and play a role in tissue remodeling. As a differentiation and activation factor, IL-6 induces B cells to differentiate and secrete antibody, it induces T cells to differentiate into cytotoxic T cells, activates cell signaling factors, and in conjunction with IL3, promotes hematopoiesis. IL-6 is prominently involved in many critical bodily functions and processes. As a result, physiological processes including bone metabolism, neoplastic transformation, and immune and inflammatory responses can be enhanced, suppressed, or prevented by manipulation of the biological activity of IL-6 in vivo by means of an antibody (Adebanjo, O. et al., J. Cell Biology 142:1347-1356 (1998)).

CNTO328 is a chimeric monoclonal antibody capable of high affinity binding to and neutralization of the inflammatory cytokine, IL-6 (U57291721). IL-6 is a potent inducer of hepatic production of hepcidin, which is the key regulator of iron homeostasis and causes anemia by blocking iron export from enterocytes and macrophages. Hepcidin is an important factor in the pathogenesis of “anemia of chronic disease”. CNTO328 treatment has previously been shown to produce profound Hb increases in Castleman's disease, a disorder caused by deregulated IL-6 production. The anemia in cancer generally, or specific cancers, may also be mediated at least in part by elevated hepcidin levels. Due to the relationship between anemia and renal cell cancer and, e.g. Castleman's disease patients, it is important to understand which of those patients having anemia will respond to treatment with an IL-6 neutralizing antibody in the face of elevated hepcidin levels and which will not.

### SUMMARY OF THE INVENTION

The invention relates to a method of treating the anemia of chronic disease, wherein the disease is selected from the group consisting or cancer and Castleman's disease, in patients exhibiting elevated levels of hepcidin using an IL-6 neutralizing antibody, thereby reducing hepcidin levels and causing hemoglobin levels to increase in the patient. In a specific embodiment, the IL-6 neutralizing antibody has a high affinity for IL-6 as determined by the KD between IL-6 and the antibody being less than 10⁽⁻⁹⁾ M. In a specific embodiment, the IL-6 neutralizing antibody is an antibody having binding regions derived from the murine antibody CLB8 and having the amino acid sequence of SEQ ID NO: 1 and 2.

The invention further relates to a method of treatment of a patient suffering from disease-related anemia having hemoglobin levels below the normal range and having a hepcidin level detectable in the serum of the patient where the disease is selected from cancer or Castleman's disease and wherein the treatment comprises the administration of an IL-6 neutralizing antibody. Thus, the determination of hepcidin in a patient suffering from disease-related anemia and the administration of an IL-6 neutralizing antibody may be used to improve the hemoglobin status of the patient which status may be unresponsive to erythropoietin receptor stimulating agent therapy due to elevated hepcidin levels.

### BRIEF DESCRIPTION OF THE SEQUENCE LISTING

| SEQ ID NO: | Description |
|---|---|
| 1 | Heavy chain variable region |
| 2 | Light chain variable region |
| 3 | Hepcidin-25 |
| 4 | Human IL-6 |

### DETAILED DESCRIPTION OF THE INVENTION

### Abbreviations

CD=Castleman's disease; ESA=erythropoiesis stimulating agents; HCV=antibody heavy chain variable region; LCV=antibody light chain variable region; MCD=multicentric Castleman's disease; Mab-monoclonal antibody

### DEFINITIONS

As used herein, an “antibody” includes whole antibodies and any antigen binding fragment or a single chain thereof. Thus, the antibody includes any protein or peptide containing molecule that comprises at least a portion of an immunoglobulin molecule, such as but not limited to at least one complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein, which can be incorporated into an antibody of the present invention. The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments to a preselected target. Examples of binding fragments encompassed within the term “antigen binding portion” of an antibody include (i) a Fab fragment, a monovalent fragment consisting of the VL, VH, CL and CH, domains; (ii) a F(ab′)2 fragment, a bivalent fragment comprising two Fab fragments linked by a disulfide bridge at the hinge region; (iii) a Fd fragment consisting of the VH and CH, domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (I 988) Science 242:423-426, and Huston et al. (1988) Proc. Natl. Acad Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Conversely, libraries of scFv constructs can be used to screen for antigen binding capability and then, using conventional techniques, spliced to other DNA encoding human germline gene sequences. One example of such a library is the “HuCAL: Human Combinatorial Antibody Library” (Knappik, A. et al. J Mol Biol (2000) 296(1):57-86).

### Cross-Reactivity.

The term “epitope” means a protein determinant capable of specific binding to an antibody. Epitopes usually consist of chemically active surface groupings of molecules such as amino acids or sugar side chains and usually have specific three-dimensional structural characteristics, as well as specific charge characteristics. Conformational and nonconformational epitopes are distinguished in that the binding to the former but not the latter is lost in the presence of denaturing solvents.

As used herein, “hepcidin” means any mammalian hepcidin or pro-hepcidin (also called LEAP (liver-expressed antimicrobial peptide)) polypeptide such as those that can be found in the GenBank database available online, including, but not limited to, accession numbers NP066998 (protein), AAH20612 (protein), AAG23966 (cDNA) and P81172 (hepcidin precursor). In particular, hepcidin means a bioactive serum form, especially hepcidin-25 (SEQ ID NO: 3).

“Humanization” (also called Reshaping or CDR-grafting) includes established techniques for reducing the immunogenicity of monoclonal antibodies (mAbs) from xenogeneic sources (commonly rodent) and for improving affinity or the effector functions (ADCC, complement activation, C1q binding). The engineered mAb can be produced using the techniques of molecular biology, using phage displayed randomized sequences, or synthesized de novo. For example, in order to construct a humanized antibody with incorporated CDR regions from a nonhuman species, the design might include variations such as conservative amino acid substitutions in residues of the CDRs, and back substitution of residues from the nonhuman mAb into the human framework regions (backmutations). The positions can be discerned or identified by sequence comparison methods, consensus sequence analysis, or structural analysis of the variable regions' 3D structure. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way or by simple sequence alignment algorithms (e.g. Clustal W), FR (framework) residues can be selected from known antibody sequences, found in such publicly accessible databases as VBASE or Kabat, and the consensus sequences optimized so that the desired antibody characteristic, such as affinity for the target antigen(s), is achieved. As the datasets of known parameters for antibody structures increases, so does the sophistication and refinement of these techniques. Another approach to humanization is to modify only surface residues of the rodent sequence with the most common residues found in human mAbs and has been termed “resurfacing” or “veneering”. A large number of both human and non-human Ig sequences are now known and freely available and used by those skilled in the art, e.g. the database and tools developed by of LeFranc et al found under the name IMGT; websites curated by the U.S. National Center for Biologics (NCBI); Kabat et al., Sequences of Proteins of Immunological Interest, U.S. Dept. Health (1983) now also greatly expanded and available online, each entirely incorporated herein by reference. Humanization or engineering of antibodies of the present invention can be performed using any method known or those developed using human immunoglobulin sequence information. Such methods are taught in, for example, Winter U.S. Pat. No. 6,982,361 and Bowdish et al. WO03/025019, the contents of which are incorporated herein by reference.

As used herein, K_{D} refers to the dissociation constant, specifically, the antibody K_{D} for a predetermined antigen, and is a measure of affinity of the antibody for a specific target. High affinity antibodies have a K_{D} of 10⁻⁸ M or less, more preferably 10⁻⁹ M or less and even more preferably 10⁻¹⁰ M or less, for a predetermined antigen. The reciprocal of K_{D} is K_{A}, the association constant. The term “k_{dis}” or “k₂”, or “k_{d}” as used herein, is intended to refer to the dissociation rate of a particular antibody-antigen interaction. The “K_{D}”, is the ratio of the rate of dissociation (k₂), also called the “off-rate (k_{off})”, to the rate of association rate (k₁) or “on-rate (kₒₙ)”. Thus, K_{D} equals k₂/k₁ or k_{off}/kₒₙ and is expressed as a molar concentration (M). It follows that the smaller the K_{D}, the stronger the binding. Thus, a K_{D} of 10⁻⁶ M (or 1 microM) indicates weak binding compared to 10⁻⁹ M (or 1 nM).

The terms “antibody”, “monoclonal antibody” or “antibody composition” as used herein refer to a preparation of antibody molecules of single molecular composition. A monoclonal antibody composition displays a single binding specificity and affinity for a particular epitope. The term also includes “recombinant antibody” and “recombinant monoclonal antibody” as all antibodies are prepared, expressed, created or isolated by recombinant means, such as (a) antibodies isolated from an animal or a hybridoma prepared by the fusion of antibody secreting animal cells and an fusion partner, (b) antibodies isolated from a host cell transformed to express the antibody, e.g., from a transfectoma, (c) antibodies isolated from a recombinant, combinatorial human or other species antibody library, and (d) antibodies prepared, expressed, created or isolated by any other means that involve splicing of immunoglobulin gene sequences to other DNA sequences. An “isolated antibody,” as used herein, is intended to refer to an antibody which is substantially free of other antibodies having different antigenic specificities.

### Overview

Human hepcidin, a polypeptide expressed predominantly by hepatocytes, is believed to be an important iron-regulating protein that negatively regulates intestinal iron absorption, iron recycling by macrophages, and iron mobilization from hepatic iron stores via the protein ferroportin. Once secreted, hepcidin binds to ferroportin and induces its internalization and degradation. Therefore, factors that trigger overproduction of hepcidin play a primary role in the pathophysiology of anemia and/or anemia of chronic disease. IL-6, in addition to hypoxia and serum iron status, is a stimulating factor for hepcidin.

Anemia is associated with a wide variety of malignancies. Cancer-associated anemia can have several etiologies, such as loss due to increased shedding of the intestinal mucosa and malabsorption of iron, internal bleeding, and increase in normal tissue distruction and neoplastic tissue iron utilization. When the body stores of iron are adequate, the stimulation of heamatopoietic cell erythropoiesis using exogenously administered ESAs can increase erythrocyte formation and correct anemia. However, in the face of increased hepcidin levels which limit iron uptake and iron mobilization from stores, ESAs may not be effective and oral iron will be poorly absorbed.

Multiple forms and stages of cancer are associated with elevated IL6 levels including; progressive breast cancer, cholangiocarcinoma, squamous cell carcinoma of the oral cavity, advanced gastrointestinal cancer, indolent non-Hodgkin's lymphoma patients, Hodgkin's disease, diffuse large cell lymphoma, small cell lung cancer, extensive disease in lung cancer, mesothelioma, metastatic melanoma, multiple myeloma (MM), ovarian carcinoma, pancreatic cancer patients especially those with weight loss, advanced stage prostate cancer, hormone-refractory and metastatic burden in prostate cancer, renal cell carcinoma particularly those with short survival time and malignant cysts (See Trikha, et al. Clin. Cancer Res., Oct. 15, 2003; 9(13): 4653-4665 for a review). The associated anemia in these patients may be as a result of an IL6 driven elevation in hepcidin levels.

The role of IL6 and its relation to serum hepcidin in the anemia of MM was studied by examining the ability of an IL6 neutralizing antibody and an IL6R neutralizing antibody to block hepcidin induction by patient derived serum in Hep3B cells in vitro (Dharma et al. 2008 Clin Cancer Res 14(1): 3262-3267). In six of 10 samples, the combination antibodies abrogated hepcidin induction. Other measurements, in this study confirmed that urinary hepcidin in the MM patients was 3-fold higher than in normal controls and, in patients without renal insufficiency, there was an inverse correlation between urinary hepcidin and hemoglobin at diagnosis.

Castleman's disease (CD) is a rare atypical lymphoproliferative disorder characterized by giant lymph node hyperplasia and angiofolicular lymph node hyperplasia with systemic manifestations such as fever, fatigue, anorexia, anemia, and wasting, particularly in patients with the plasma-cell or mixed-type variants of the disease (Nishimoto, et al. 2005 Blood 106(8):2627-2632). Hepatosplenomegaly, lymph node enlargement, and multiple abnormalities associated with liver function such as hypoalbuminemia and hypocholesterolemia are also common

The mulitcentric form of CD exhibits symptoms similar to lymphoma (cancer of the lymph nodes). Patients with multicentric CD have limited treatment options, including steroids, combination chemotherapy and immunomodulatory agents, which all have variable response rates. Dysregulated IL-6 production by germinal center B cells has been associated with clinical symptoms and biochemical abnormalities in patients with CD. CD patients may also be HIV-positive. Overproduction of a viral analogue of IL-6 has also been implicated in the pathogenesis of CD. There is a continued unmet medical need for effective therapy to manage CD patients.

Effective therapies, particularly those not associated with other unwanted side effects such as with glucocorticosteroid use, that control disease and/or pathogenic sequelae such as anemia by targeting the underlying mechanisms responsible for the symptoms and deterioration in body functions is still needed.

Iron stores or total body iron status, and inflammation, represent upstream pathways of hepcidin regulation. Therefore, serum transferrin receptor, transferrin saturation, and CRP are indirect markers related to iron-moblization and erythropoietic activity; where transferrin receptor and transferrin saturation status relate to the availability of iron for heme synthesis and serum CRP levels relates most directly to IL-6 produced in response to inflammation or malignant growth process and therefore to hepcidin production. The inter-relationship between these factors may define the propensity for anemia to occur and the etiology, however, the precise relationship has not been demonstrated for any given disease to date, particulary, in diseases of the kidney where hepcidin elimination may be effected. Renal cell carcinoma as discussed above is a cancer associated with elevated IL6 levels.

Preliminary results in patients with Castleman's disease have shown that blocking IL-6 binding to the IL6 receptor using either an anti-IL-6 antibody, BE-8 (Beck et al. New Eng J Med 330(9): 602-605, 1994) or CNTO328 (vanRhee et al. and Example 1) or a neutralizing antibody to IL6 receptor, MRA, formerly called rhPM-1 (Sato, et al. Cancer Res. 1993; 53:851-856; Nishimoto, et al. Blood 95: 56-61, 2000; Nishimoto, et al. Blood, Oct. 15, 2005; 106(8): 2627-2632) is accompanied by an increase in hemoglobin. Preliminary results also showed that CNTO328 increases hemoglobin in renal cell cancer patients (Schipperus et al. J Clin Oncol 27, 2009 (suppl; abstr e20648). Addition information on hepcidin levels in 11 newly diagnosed patients with solid tumors or malignant lymphoma, excluding those showing myelosuppression or hepatic or renal failure, indicated that hepcidin levels were averaged 46.1 ng/ml while normal was 1.4 ng/ml with IL-6 greatly elevated and Hb slightly suppressed (weak negative correlation of Hb with 11-6 and hepcidin) Takahashi et al. J Clin Oncol 27, 2009 (suppl; abstr e20655).

Recently a validated immunoassay for hepcidin has become available (Ganz, et al. Blood, Nov. 15, 2008; 112(10): 4292-4297) as well as a radioimmunoassay (British Journal of Haematology, 146, 317-325).

The question of whether treatment with an agent capable of neutralizing IL6 activity, such as CNTO328, is effective in lowering hepcidin levels and therefore ameliorating the anemia exhibited by cancer patients exhibiting higher than normal levels of hepcidin, such as CD and renal cell carcinoma patients, was addressed by applicants. The present inventors measured baseline hepcidin levels in RCC patients and then again eight days after CNTO328 treatment along with changes in Hb levels. The administration of CNTO328 markedly reduced serum hepcidin and increased Hb in 92% of patients treated.

### Anti-IL-6 Antibodies

Preferred antibodies of the present invention include those chimeric, humanized and/or CDR grafted antibodies that will competitively inhibit in vivo binding to human IL-6 of anti-IL-6 murine CLB-8, chimeric anti-IL-6 CLB-8, or an antibody having substantially the same binding characteristics, as well as fragments and regions thereof. In addition, when bound to IL6, the antibody will prevent IL6 from activating the IL6 receptor complex. The IL6 receptor consists of an 80 kD binding sub-unit, IL6Rα and the signal transduction sub-unit, gp130. IL6 binds to the IL6Rα sub-unit and initiates the association of IL6Rα and gp130 resulting in a high affinity receptor and signal transduction leading to, e.g. phosphorylation of STAT3. IL6Rα also exists in a soluble form. IL6 can bind to soluble IL6R (sIL6R) and the complex can act on cells expressing gp130.

Preferred antibodies of the present invention are those that bind an epitope recognized by CLB-8 and cCLB-8, which are included in the Site I epitope as described by Brackenhoff et al. (J. Immunol. (1990) 145: 561-568). Preferred methods for determining monoclonal antibody specificity and affinity by competitive inhibition can be found in Harlow, et al, Antibodies: A Laboratory Manual, Cold Spring Harbor Laboratory Press, Cold Spring Harbor, N.Y., 1988), hereby incorporated by reference into the present application. The antibody of the invention binds at least one specified epitope specific to human IL-6 protein, subunit, fragment, portion or any combination thereof, to which CNTO328 or its parent molecule, CLB-8 monoclonal antibody, binds. The epitope can comprise at least one antibody binding region to which the CLB-8 antibody binds, which epitope is preferably which is comprised of amino acids Gln29-Leu34 in close proximity of the carboxyl terminus of the IL-6 molecule as represented by SEQ ID NO: 4. Kalai, M, et al. (Eur. J. Biochem. 249, 690-700 (1997) showed that the parent antibody of CNTO328, also called CLB IL-6/8, recognized amino acid residues crucial for the binding of IL-6 to the IL-6R (gp80). These studies also indicated that its epitope covers the ends of both the AB loop and the D helix regions of the IL-6 molecule.

The anti-human IL-6 antibody can further bind IL-6 with an affinity (K_{D}) of at least 10⁻⁹ M, preferably at least 10⁻¹⁰ M, and substantially neutralize at least one activity of IL-6 protein in vivo such as inhibit of activation of the IL6 receptor and phosphorylation of STAT3. In a preferred embodiment, the antibody binds IL-6 with an affinity (K_{D}) of at least 5×10⁻¹¹ M, preferably 1×10⁻¹¹ neutralizes human IL-6.

### Methods of Assessing Changes in Serum Factors

Human hepcidin is encoded as an 84-amino acid prepropeptide containing a typical N-terminal 24-amino acid endoplasmic reticulum-targeting signal sequence, and a 35-amino acid proregion with a consensus furin-cleavage site immediately followed by the C-terminal 25 amino acid bioactive iron-regulatory hormone, human hepcidin-25 (SEQ ID NO: 3). Subsequent cleavage of the new N-terminus of this bioactive peptide results in truncated forms of human hepcidin, such as human hepcidin-20 (i.e., amino acids 6-25 of SEQ ID NO: 3) and human hepcidin-22 (amino acids 4-25 of SEQ ID NO: 3) also found in vivo. It is, however, the N-terminal portion of hepcidin-25 which has been shown to bind to the ferroportin receptor.

An argument may be made that, depending on the epitope bound by the detection antibody in, e.g. an EIA, the assay will not differentiate the active, physiologically relevant forms of human hepcidin capable of activating Fpn, from inactive, species (see, for example, Kemna, E. H., et al, Haematologica, 93(1):90-7 (2008)). Assay for hepcidin-25 by LC/MS (liquid chromatography/mass spectroscopy) results in the separation of the various forms of hepcidin (see, for example, (Gutierrez, J. A., et al., BioTechniques, 38:S13-S17 (2005), Murphy, et al., Blood, 110:1048-54 (2007) and Kemna, E. H., et al., Clin. Chem. 53:620-8 (2007). An anti-hepcidin-25 specific antibody has now been reported (US 20090136495).

Hepcidin may also be quantitated using an isotope dilution micro-HPLC-tandem MS (MS/MS) method that allows the quantification of hepcidin-25 present at less than nmol/L concentrations (Roche Diagnostics)

Commercially available immunoassays for human hepcidin are now available. A particularly useful assay is an ELISA offered by Intrinsic Lifesciences, San Diego, Calif. (Ganz, et al. Blood, Nov. 15, 2008; 112(10): 4292-4297).

CRP levels and other standard clinical parameters such as albumin, transferrin, transferrin saturation, homocysteine, serum amyloid A (SAA), coeruloplasmin, IL-6 related proteins such as sIL-6R, leukemia-inhibiting factor (LIF), oncostatin M (OSM), ciliary neurotropic factor (CNTF): or other acute phase proteins such as IL1alpha/beta, TNFalpha, alpha1-anti-chymotrypsin, alpha1-macroglobulin, insulin, and complement component C3, and other inflammatory proteins, such as fibroblast growth factor beta (FGFbeta), transforming growth factor beta (TGFbeta), and cytokines or interferons are measured by standard techniques known in the art.

Methods of Treatment with Anti-IL-6 Antibody

Clinical trials using monoclonal antibodies against IL-6 have been conducted in multiple diseases including plasma cell leukemia (Klein, B. et al. 1991 Blood 78 (5): 1198-1204), multiple myeloma, Castleman's disease (Beck et al. New Eng J Med 330(9): 602-605, 1994;), rheumatoid arthritis, renal carcinoma, and AIDS associated lymphoma (see Trikha et al 2003 Clin. Cancer Res. 9(13): 4653-4665 for a review of the early work).

CNTO328 has been reported for treatment of multiple myeloma (van Zaanen et al. 1996 J. Clin. Investig., 98: 1441-1448; van Zaanen et al. 1998 Br. J. Haematol 102: 783-790) and Castleman's disease (Van Rhee et al., 2008 November 2008; Blood 112: 1008). CNTO328 may be used to treat a variety of malignancies and lymphoproliferative disorders where IL6 is known to be elevated or play a role in the pathogenesis of the disease and related symptoms including but not limited to anemia, cachexia, anorexia, disturbances in iron metabolism, bone resorption, fever, sweating, and edema; as described herein or known in the art.

The IL6-neutralizing antibody which is CNTO 328 and/or comprised of SEQ ID NO: 1 and 2, may be administered by intravenous infusion or sub cutaneously or, using appropriate formulations and concentrations by any route whereby a safe and effective dose is received by the subject and appropriate serum, tissue or other body compartment levels for effective and safe treatment may be accomplished. Efficacy can be monitored by a variety of criteria as taught herein and known in the art such as reduction in tumor mass, increase in hemoglobin levels, reduction in serum levels of markers (sometimes called biomarkers), indicating a change in the volume, function, or activity of a cell, tissue or organ in the patient receiving the IL6-neutralizing antibody. Examples of serum markers useful in the invention include: hemoglobin, hepcidin, IL6, CRP, transferrin, ferritin, soluble transferring receptor, transferrin saturation status, total iron binding capacity, homocysteine, and alpha1-microglobulin.

The dose of CNTO328 given in each administration will typically range from 0.1 to 50 mg/Kg, more typically from 1 to 20 mg/Kg, and, where appropriate from 3 to 13 mg/Kg. The dosing may be continuous, multiple times per day, once per week or on a monthly basis.

Treatment of a subject with CNTO328 or a related anti-IL-6 antibody, fragment, or variant can further comprise administration of one or more additional agents prior to, in conjunction with, subsequently to, or in alternating courses of any suitable and effective amount of a composition or pharmaceutical composition deemed appropriate for the benefit of the subject. For example, treatment of malignant or lymphoproliferative disorders may be accompanied by treatment with a glucocorticosteroid drug such as prednisone or dexamethasone. Examples of other agents known now or to be developed or recognized appropriate for the treatment of IL6-associated diseases are included as potentially useful in conjunction with the use of CNTO328 to reduce hepcidin levels in patients in need thereof.

### Example 1

### CNTO 328 AS A SINGLE AGENT IN CASTLEMAN′S DISEASE

CNTO 328 was used in Castleman's patients in an open-label, nonrandomized, dose-finding Phase 1 study in subjects with hematological malignancies including subjects with CD. The objectives of the study were to assess the safety, pharmacokinetics, pharmacodynamics, immune response, and clinical effects of multiple dosing regimens of CNTO 328 administered as an intravenous (IV) infusion in subjects with CD. CD subjects enrolled in each cohort are shown below:

TABLE 1

| | |
|---|---|
| Cohort 1 (n = 1) | 3 mg/kg administered IV over two hours every 2 |
| | weeks (increased to 6 mg/kg Q2 weeks after dose 11). |
| Cohort 2 (n = 2) | 6 mg/kg administered IV over two hours every 2 weeks |
| Cohort 3 (n = 6) | 12 mg/kg administered IV over two hours every 3 weeks |
| Cohort 4 (n = 3) | 6 mg/kg administered IV over two hours every week |
| Cohort 5 (n = 3) | 12 mg/kg administered IV over two hours every 2 weeks |
| Cohort 6 (n = 2) | 12 mg/kg administered IV over one hour weekly |
| Cohort 7 (n = 6) | 9 mg/kg administered IV over one hour every 3 |
| | weeks (increased to 6 mg/kg Q2 weeks after dose 11) |

For simplicity, the results are presented in 3 groups (6 mg/kg (including 1 subject in 3 mg/kg q 3 mg/kg q 2 wks), 9 mg/kg and 12 mg/kg)

### Key Inclusion Criteria:

The Castleman's disease subjects must have been 18 years or older and have had either localized disease that in the opinion of the investigator is not amenable to surgical resection or multicentric disease. Subjects must have had active, symptomatic disease that in the opinion of the investigator requires therapy. The subject must have had adequate bone marrow, liver, and renal function as evidenced by an absolute neutrophil count ≧1.0×10⁹/L, platelets ≧75×10⁹/L; without transfusion dependency, and liver functions tests gauged for patients with and without liver metastases or with bone involvement. Hemoglobin was ≧8.0 g/dL (5 0 mmol/L; 80 g/L); without transfusion dependency, for Cohort 7, hemoglobin ≧7.5 g/dL (4.7 mmol/L; 75 g/L).

Clinical Benefit Responses (CBR) is defined as shown improvement at least in one of the following six measures and is at least stable for the remaining measures:
1. 2 g/dL increase in hemoglobin (Hb) compared with baseline without transfusions,
2. 1 CTC grade decrease in fatigue,
3. 1 CTC grade decrease in anorexia,
4. fever/night sweats: decrease at least 2° C. compared to baseline or return to 37° C.,
5. 5% or more increase in weight, or
6. 25% decrease bidimensionally in size of largest lymph node.
   Tumor responses are determined based on independent radiological review. Tumor response is based on modified Cheson criteria (1999) defined as the following:

TABLE 2

| | |
|---|---|
| Complete Response (CR) | complete disappearance of |
| | all measurable and evaluable |
| | disease (eg, pleural effusion). |
| Partial Response (PR) | ≧50% decrease in sum of product |
| | of diameters (SPD) of |
| | indicator lesion(s), with at least |
| | stable disease (SD) in all |
| | other evaluable disease. |
| Stable Disease (SD) | failure to attain CR or PR, without |
| | evidence of progressive disease (PD) |
| Progressive Disease (PD) | ≧50% increase in SPD of |
| | indicator lesion(s) compared to |
| | nadir or at least 1 new lesion |
| | that has been confirmed and |
| | measures >1.5 cm in longest |
| | dimension. Malignant |
| | transformation in a previously |
| | defined mass will also be |
| | considered PD. |

TABLE 3
Summary of duration of exposure

| | 6 mg/kg | 9 mg/kg | 12 mg/kg | Combined |
|---|---|---|---|---|
| | (N = 6) | (N = 6) | (N = 11) | (N = 23) |
| Time from 1st to final | 295.0 | 178.0 | 575.0 | 331.0 |
| CNTO 328 | (43.0- | (1.0- | (57.0- | (1.0- |
| administration (days) | 1148.0) | 300.0) | 865.0) | 1148.0) |
| Median, range | | | | |
| Total number of CNTO | 26.5 | 9.5 | 36.0 | 17.0 |
| 328 administrations | (7.0- | (1.0- | (4.0- | (1.0- |
| Median, range | 107.0) | 15.0) | 42.0) | 1070.0) |

### Results

The overall Clinical Benefit Response results are shown below (Table 5).

TABLE 4

| | 6 mg/kg | 9 mg/kg | 12 mg/kg | Combined |
|---|---|---|---|---|
| Characteristic | (N = 6) | (N = 6) | (N = 11) | (N = 23) |
| Hemoglobin, n (%) | 3 (50.0) | 2 (33.3) | 4 (36.4) | 9 (39.1) |
| Fatigue, n (%) | 4 (66.7) | 4 (66.7) | 10 (90.9) | 18 (78.3) |
| Anorexia, n (%) | 2 (33.3) | 3 (50.0) | 5 (45.5) | 10 (43.5) |
| Fever/night | 3 (50.0) | 3 (50.0) | 6 (54.5) | 12 (52.2) |
| sweats, n (%) | | | | |
| Weight, n (%) | 3 (50.0) | 0 (0.0) | 7 (63.6) | 10 (43.5) |
| Size of largest | 4 (66.7) | 2 (33.3) | 10 (90.9) | 16 (69.6) |
| lymph node, n (%) | | | | |

### Study Results Summary

Tumor response frequency (CR+PR) was confirmed by independent radiologic review. At the highest (12 mg/kg) dose level, 8 of 11 (73%), of evaluable subjects treated with time to initial best-response ranging from 57 to 337 days. The subjects demonstrated a high frequency of clinical benefit response (18 of 23 subjects, 78%) with 100% CBR response rate achieved in the 12 mg/kg group.

Improvement in hemoglobin by 1 g/dL was seen in a majority, 19 of 23 subjects, with median maximum increase of 2.1 g/dL (0.2 to 7.2 g/dL). Complete suppression of C-reactive protein (CRP), a surrogate for IL-6 activity, was demonstrated in 78% of subjects for whom post-baseline values were obtained. This was particularly noteworthy in view of the markedly elevated baseline CRP levels seen in the study population (median 23.4 mg/L [range 1 to 260 mg/L]).

CNTO 328 produced a favorable safety profile even after prolonged exposure in this study. Based on data showing that the surrogate marker for IL-6, CRP, was completely suppressed in this CD patient group and evidence showing IL6 stimulates hepcidin production, the marked improvement in hemoglobin status by 2 g/dL in 39% of patients and by 1 g/dL in the majority (83%) of patients may involve the reduction of hepcidin.

### Example 2

### CNTO 328 TREATMENT IN RENAL CELL CARCINOMA DECREASES HEPCIDIN LEVELS

A retrospective study of 38 RCC patients treated with CNTO328 was done to assess whether treatment was associated with a decrease in hepcidin.

Patients and Methods: Serum Hb, IL-6 and CRP (a surrogate for IL-6 activity) levels were prospectively studied in RCC patients selected from two 6 mg/kg CNTO328 treatment cohorts (IV Q2W or Q3 W) in a phase 1/2 study. Free and total IL-6 could not be measured due to interference of the drug with assay performance which prevents accurate measurement. Serum hepcidin levels were retrospectively measured using a hepcidin C-ELISA. The change from baseline in Hb, hepcidin and CRP levels was calculated at multiple time points. The association between Hb response (defined as max Hb increase of ≧1 g/dL) and change in hepcidin and CRP levels was evaluated by Pearson Correlation Coefficient (r).

Results: The 38 RCC patients had a median baseline Hb of 13.2 g/dL (10.1-17.1) which is considered moderately anemic. All patients had normal renal function throughout the study. Two patients were excluded from analysis because of blood transfusions at baseline. None of the patients studied received ESAs or blood transfusions during screening or treatment. Treatment with CNTO328 resulted in an Hb increase during study in 35 (92%) of the 38 patients starting on Day 8, with 25 (66%) achieving a max Hb increase of 1 g/dL (median 2.0; range 1.0-3.5).

Hb responses were not due to tumor response, were independent of dosing schedule (Q2W vs Q3W) and were even seen in 6 (86%) out of 7 patients with a baseline Hb <12 g/dL. As expected, a marked reduction in day 8 hepcidin was noted (median decrease of 61.1%, range −90% to −53.9%). A moderate correlation was found between Hb response and the Day 8 percent change in hepcidin (r=−0.56, n=19) but not between baseline hepcidin and Hb response (r=0.056, n=21). A sustained suppression of serum CRP levels from baseline was evident in all patients and Hb responses were moderately correlated with the Day 8 absolute change in CRP (r=−0.41, n=24) and less well correlated with baseline CRP (r=0.31, n=25) levels. Max Hb levels (median 14.9 g/dl, range 11.1-17.7) did not exceed the upper limit of normal for any patient, and no thromboembolic events were observed.

Conclusion: CNTO328 was associated with normalization of Hb in this moderately anemic RCC population, presumably due to reduction of hepcidin via IL-6 blockade. Thus, treatment with CNTO328 in the absence of ESAs, may provide an alternative to other methods of adjunctive care to manage anemia such as transfusions in renal cell cancer patients having anemia. Hb response correlated with hepcidin and CRP level reduction in this study.

| | CNTO 328, | CNTO 328, | |
|---|---|---|---|
| | 6 mg/kg | 6 mg/kg | |
| | Q2W | Q3W | Combined |
| Number of patients | 19 | 19 | 38 |
| Median age | 62 | 57 | 60 |
| (range), yrs | (50-77) | (26-82) | (26-82) |
| Males | 18 | 16 | 34 |
| Median baseline Hb | 13.9 | 12.5 | 13.2 |
| (range), g/dL | (12.2-17.1) | (10.1-16.4) | (10.1-17.1) |
| Subjects with an | 16 | 19 | 35 |
| increase in Hb | (84%) | (100%) | (92%) |
| Median increase in | 1.9 | 1.3 | 1.7 |
| Hb from baseline | (0.4-3.5) | (0.2-3.2) | (0.2-3.5) |
| (range), g/dL | | | |
| Median Max Hb | 15.1 | 14.3 | 14.9 |
| during treatment | (14.2-17.7) | (11.1-16.6) | (11.1-17.7) |
| (range), g/dL | | | |
| Median baseline | 69.1 | 109.1 | 84.0 |
| hepcidin (range), | (43.8-217.0) | (5.0-510.5) | (5.0-510.5) |
| ng/mL | | | |
| Median Day 8 | −43.7 | −67.2 | −61.1 |
| change in hepcidin | (−72.3-+53.9) | (−90.0-0) | (−90.0-+53.9) |
| (range), % | | | |

What is to be claimed:

1. A method of reducing the level of serum hepcidin in a subject suffering from one or more clinical abnormalities associated with IL6 production in a malignancy or lymphoproliferative disorder, wherein the methods comprises the administration of a high affinity IL6 neutralizing antibody comprising SEQ ID NO: 1 and 2.

2. A method of claim 1 wherein the clinical abnormality is selected from the group consisting of decreased hemoglobin, fever or night sweats, anorexia, weight loss, and lymph node enlargement.

3. A method of claim 2 wherein the clinical abnormality is a less than normal hemogloblin level.

4. A method of claim 3 wherein the hemogloblin level prior to treatment with the antibody is less than 12 g/dL.

5. A method of any one of claims 1-4 wherein the hepcidin level in the patient is reduced by 10-90%.

6. A method of claim 5 wherein the hepcidin level is reduced by 10-90% within 8 days after administration of the antibody.

7. A method of claim 1 wherein the antibody is CNTO 328.

8. A method of claim 1 wherein the IL6-associated disease is selected from the group consisting of renal cell carcinoma, Castleman's disease, multiple myeloma, and advanced prostate cancer.

9. A method according to claim 1, wherein the ability of the antibody to neutralize IL6 bioactivity is demonstrated by suppression of baseline serum CRP levels.

10. A method according to claim 1, wherein the IL-6 antibody comprises one heavy chain or light chain variable region comprising SEQ. ID Nos. 1 or 2.

11. A method according to claim 1, wherein the IL-6 antibody binds IL-6 with an affinity (Kd) of at least 10-10 M.

12. A method according to claim 1, wherein the IL-6 antibody binds IL-6 with an affinity (Kd) of at least 10-11 M.

13. A method according to claim 1, wherein the IL-6 antibody binds IL-6 with an affinity (Kd) of at least 10-12 M.

14. The method according to any of claims 1, in which the monoclonal antibody competes with the antibody comprising SEQ ID NO: 1 and 2 for binding to human IL6.

15. The method according to claim 1, in which the monoclonal antibody is administered intravenously

16. The method according to claim 1, in which the monoclonal antibody is administered in the amount of from 3 mg/kg to 12.0 mg/kg body weight.

17. The method according to claim 1, in which the monoclonal antibody is administered in a one hour infusion of said antibody.

18. The method according to claim 1, in which the monoclonal antibody is administered in a two hour infusion of said antibody.

19. The method according to claim 1, in which the subject is a human patient exhibiting multicentric Castleman's disease.

20. The method of claim 1, which comprises administering a corticosteroid in combination with an IL-6 antagonist.
